(19) United States
(12) Patent Application Publication
(10) Pub. No.: US 2005/0053626 A1
(43) Pub. Date: Mar. 10, 2005

(54) Grace process biotechnology patent the cure to the herpes virus

(76) Inventor: John Ames Harrington, Hollywood, FL (US)

Correspondence Address: JOHN AMES HARRINGTON, 1839 WASHINGTON ST., HOLLYWOOD, FL, 33020, US

(21) Appl. No.: 10/446,200

(22) Filed: Sep. 5, 2003

(51) Int. Cl. C12Q 1/70; A61K 39/275; A61K 39/285

(52) U.S. Cl. 424/232.1; 435/5

(57) ABSTRACT

Step 1. Method of treating disease: this procedure must be done by a certified Medical Doctor, who has access to controlled substances. Doctor is to give patient one normal smallpox vaccination. Do not touch or itch the blister it causes, and when the blister scabs, continue not to touch. Wait until the scab naturally falls off, return to same Doctor as soon as possible to repeat Step 1. within 24 to 48 hours, it is imperative. Repeat process over and over, each time the process is done, the blister and scab gets smaller and smaller until the process is done, and there is no visible blister or scab. This may take 4 to 6 weeks for process to be complete; process longer in severe cases. To save on scaring it is recommended to vaccinate patients on the same site where original childhood smallpox vaccination was administered. This is the total complete cure to the Herpes Virus. Patient becomes immune to the virus. Virus is contagious until the GRACE PROCESS is complete.

### TECHNICAL DISCLOSURE

Smallpox Vaccination Series 770

Step 1. Method of Treating Disease:

This procedure must be done by a certified doctor, who has access to control substances (vaccines). Doctor is to give patient 1 normal smallpox vaccination. Do not touch or itch the blister caused by vaccination, when blister scabs, continue not to touch. Wait until scab naturally falls off, return to same doctor as soon as possible to repeat Step 1. Within 24 to 48 hours, it is imperative. Repeat Step 1. {Smallpox Vaccination Series 770}, each time the patient is vaccinated the blister & scab get smaller & smaller until the patient is vaccinated & there is no visible blister or scab. This may take 4-6 weeks for process to be complete, process longer in severe cases. To save on scaring it is recommended to vaccinate on the same site where original childhood smallpox vaccination was administered. This method of treatment win eradicate the Herpes Virus. Type 1 & 2. On male or female. Patient becomes immune to the Virus when the Smallpox Vaccination Series 770 is completed. Virus is contagious until Smallpox Vaccination Series 770 is completed.

Humoral Immune Response. Body was uninterrupted, no fever, no side effects, no dry mouth, body fluids & functions normal during the {Smallpox Vaccination Series 770}. Expect for very extremely itchy vaccination site, itching around the site is permitted only, do not touch it is contagious.

1. Smallpox is directly related to Herpes Type 1 & 2.

2. The DNA Virus Family of {(Poxviridae) (Smallpox Virus)}, Vaccina Virus Herpesviridae Virus Herpes.

3. The Sub-Family of Poxviridae is Chorodopoxvirinae.

4. Treating patients with a Smallpox vaccination series 770 as explained in Technical Disclosure will eradicate the Herpes Virus Type 1 & 2.

5. In 1974, I was diagnosed with a severe case of Genital Herpes.

6. In 1974, I discovered the family relationship between Smallpox & Herpes.

7. In 1974, I invented a treatment for Genital Herpes, using the Smallpox vaccination in series on myself as the first patient.

8. During the Smallpox Vaccination Series 770, in 1974 I immediately started to feel better and the Herpes sores disappeared to this date Jun. 19, 2004.

9. I ended the Smallpox Vaccination Series after I was vaccinated & there was no blister or visible reaction.

10. The body becomes immune to the Herpes Virus after the Smallpox Vaccination Series 770 is complete.

11. Patients who have had a Smallpox Vaccination earlier in life, are safe to use the Smallpox Vaccination Series 770.

12. Patients who have never had a Smallpox Vaccination earlier in life the mortality rate is as normal.

13. The Smallpox Vaccination Series 770 takes 4 to 6 weeks to be complete, longer in severe cases, and is contagious until is complete.
